(19) United States
(12) Patent Application Publication
(10) Pub. No.: US 2002/0160010 A1
(43) Pub. Date: Oct. 31, 2002

(54) USE OF PREPARATIONS CONTAINING ANTI-CD44 ANTIBODIES IN THE TREATMENT OF CERTAIN TUMOURS AND THE SUPPRESSION OF IMMUNE REACTIONS

(76) Inventors: PETER HERRLICH, KARLSRUHE (DE); HELMUT PONTA, WEINGARTEN (DE); JAN SIMON, MERZHAUSEN (DE); JOHANNES WEISS, WALDKIRCH (DE)

Correspondence Address: STERNE KESSLER GOLDSTEIN & FOX, 1100 NEW YORK AVENUE NW, SUITE 600, WASHINGTON, DC, 200053934

(21) Appl. No.: 09/380,578

(22) PCT Filed: Feb. 26, 1998
(86) PCT No.: PCT/EP98/01089

(30) Foreign Application Priority Data
Mar. 4, 1997 (DE) 19708713.2

(51) Int. Cl. A61K 39/395; A61K 39/00; A61K 39/38; C07K 2/00; C07K 4/00; C07K 5/00; C07K 7/00; C07K 14/00; C07K 16/00; C07K 17/00; A61K 38/00; C12P 21/08

(52) U.S. Cl. 424/184.1; 424/130.1; 424/145.1; 424/158.1; 424/138.1; 530/300; 530/387.1; 530/388.8

(57) ABSTRACT

The present invention relates to the use of anti-CD44 antibodies of both the constant (sDC44) and the variable part of CD44 (vCD44) for treating certain tumours and other diseases associated with the degeneration and activation of Langerhans cells (LC) and dendritic cells (DC) inside a mammalian body, including humans, and for treating undesirable immune reactions.

The invention further relates to an ex vivo cultivation process for producing dendritic cells.

The invention relates to the use of anti-CD44 antibodies against both the constant and the variable part of CD44 for treating specific tumours and other diseases associated with the degeneration and activation of Langerhans cells (LC) and dendritic cells (DC) inside a mammalian body, including human beings, and for treating unwanted immune reactions. The invention further relates to an ex vivo culture process for preparing dendritic cells.

Epidermal Langerhans cells (LC) belong to the family of the dendritic cells (DC) of the blood which are an important part of the peripheral immune system (Simon, J. C. et al., Hautarzt 43:241-249, 1992). Because of their exposed position in the skin or other peripheral organs they form the “sentries of the immune system” (Simon, J. C. et al., 1992, loc. cit.). They are among the most potent immune cells in the mammal or in the human body and are the only ones capable of triggering primary T-cell-mediated immune reactions. Examples of such immune reactions are allergies of the delayed type, transplant rejections and immune responses to viruses or tumours (Grabbe, S. et al., Immunology Today 16:117-121, 1995; Moll, H., The immunefunctions of epidermal Langerhans cells, Heidelberg: Springer Verlag, 1995; Steinmann, R. M. et al., Adv. Exp. Med. Biol. 329:1-9-, 1993; Schuler, G. et al., Adv. Exp. Med. Biol. 329: 243-249, 1993).

The prerequisite for this immune function of the LC/DC is their migration from the skin or from other organs into the peripheral lymph nodes (Romani, N. et al., J. Exp. Med. 180:83-93, 1994; Kripke, M. L. et al., J. Immunol. 145:2833-2838, 1990; Cumberbatch, M. et al., Immunology 81:395-401, 1994). There, they accumulate in distinct anatomical regions, the so-called paracortical T-cell-areas, where they activate antigen-specific resting “naive” T-lymphocytes. The mechanisms which block this controlled migration and accumulation are unknown.

On account of their exception ability to induce immune responses, dendritic cells prepared outside the body have recently also been used as immunotherapeutic agents, e.g. for treating certain tumours (Grabbe, S. et al., 1995, loc. cit.) . However, the DCs prepared by the known process only rarely reach the peripheral lymph nodes in which they are capable of triggering an immune response, which means that the DCs prepared ex vivo according to the prior art are not effective immunotherapeutic agents to the extent one would wish.

The surface glycoprotein CD44 is of critical importance for the migration of activated immune cells and certain tumour cells into the peripheral lymph nodes (Zahalka, M. A. et al., J. Immunol. 154:5345-5355, 1995; Jalkanen, S. et al., J. Cell. Biol. 105:983-990, 1987; Seiter, S. et al., J. Exp. Med. 177:443-455, 1993; Thomas, L. et al., J. Invest. Dermatol. 100:115-200, 1993; Thomas, L. et al., J. Cell. Biol. 118:971-977, 1992). It is not known whether CD44 and/or its isoforms play a part in the migration of LC and DC and their accumulation at lymph nodes and their immune function.

The DNA- and amino acid sequence of the constant form or standard form of CD44 in humans and various animals are described, for example, in Tölg, C. et al., Nucl. Acids Res. 21:1225-1229, 1993; Screaton, G. R. et al., Proc. Natl. Acad.- Sci. USA 89:12160-12164, 1992; Stamenkovic, I. et al., The EMBO Journal 10:343-348, 1991; Günthert, U. et al., Cell 65:13-24, 1991; Stamenkovic, I. et al., Cell 56:1057-1062, 1991).

CD44 is a glycoprotein located on the cell surface, which was originally described as “lymphocyte homing receptor” (Screaton, G. R. et al, 1992, loc. cit.). It is thought to be involved in the adhesion of lymphocytes to specific mucosal endothelial cells of veins (Peyer's patch or Peyer plaques or Folliculi lymphatici aggregati) or post-capillary veins in the lymph nodes (Jalkanen S. T. et al., Eur. J. Immunol. 16:1195-1202,1986; Camp, R. L. et al., J. Exp. Med. 173:763-766,1991). Moreover, the CD44 glycoprotein is thought to be involved in the maturation and activation of lymphocytes or to have a (co-)determining effect on the increased migration capacity of all lymphoblasts (e.g. Camp, R. L. et al., 1991, loc. cit; Huet, S. et al., J. Immunol. 143:798-801, 1989) and is supposed to act as an anchorage point for other adhesion molecules (Shimizu, Y. et al., J. Immunol. 143:2457-2463, 1989). However, as of now, not all these functions of CD44 have been clearly explained.

It has been found in rat tumour cells which metastasise via the lymphatic system (BSp73 cells of a spontaneous rat pancreas adenocarcinoma) that these cells express variants of CD44 (vCD44) and are responsible for the “trafficking” of tumour cells. These findings have also been demonstrated on other tumour cell lines.

It has also been shown that vCD44 glycoprotein imparts metastasising properties to a tumour which was a priori not metastasising, whereas the standard form of CD44 (sCD44) is not capable of this. Thus, it can now be assumed that vCD44 compared with sCD44 is a metastasis-specific protein which gives tumours the ability of metastasising through the lymphatic channels (Günthert, U. et al., 1991, loc. cit.) .

Further clarification of the vCD44 glycoprotein in the rat up to the final characterisation of the DNA- and amino acid sequence were provided by Günthert, U. et al., 1991, loc. cit., by means of the BSp73-rat cell system which consists of two morphologically or phenotypically different syngenic cell variants: a non-metastasising variant AS (BSp73AS) and a metastasising variant ASML (BSp73ASML) (Matzku, S. et al., Cancer Research 49:1294-1299, 1989).

For this purpose, monoclonal antibodies (mAbs) were prepared which recognise the antigenic determinant on the metastasising variant BSp73ASML.

Cell lines were obtained both from the primary tumour (subcutaneous non-metastasising node consisting of BSp73AS-cells) and also a metastasis thereof (BSp73ASML-cells which metastasise into lymph nodes and lungs). mAbs were prepared which are directed against the membrane proteins of BSp73ASML-cells (Matzku, S. et al., 1989, loc. cit.). One of these mAbs, which recognises only epitopes on BSp73ASML-cells but not those on BSp73AS-cells or other non-tumourigenic cells, was used to search an E. col cDNA-expression library prepared from poly(A) ⁺RNA from BSp73ASML-cells and a suitable vector system. In this way it was possible to identify a clone (pMeta-1) which contains the total cDNA with a length of 3207 bp and which codes for an additional domain of 162 amino acids. This domain cannot be found either in sCD44-cells or in other non-metastasising tumour cells and contains the mAb specific epitope-coding region. Using mRNA preparations from cells of different tissues and mRNA:DNA hybridisations carried out with them with various DNA probes obtained from the cDNA clones it was found that vCD44 is a splice variant of sCD44 and that the expression of vCD44 RNAs is associated with the formation of metastases. It is thus found that the additional extracellular domain (amino acids 224 to 385 in pMeta-1) coded by the 486 bp long insert is the part of the surface glycoprotein vCD44 which is relevant to metastases (Matzku, S. et al., 1989, loc. cit.).

The metastatic tumour growth (adenocarcinoma in the rat) was able to be suppressed after immunisation with monoclonal antibodies which recognise the above-mentioned epitope or which react specifically with this extracellular region of vCD44 (Reber, S. et al., Int. J. Cancer 46:919-927, 1990).

Identification of this variant extracellular domain in the rat (pMeta-1 or rMeta-1) made it possible to clarify the equivalent human nucleotide and amino acid sequences as well:

It was recently shown that the expression of variants of the surface glycoprotein CD44 is necessary and sufficient to trigger so-called spontaneous metastatic behaviour both in a non-metastasising pancreas-adenocarcinoma-cell line in the rat and also in a non-metastasising fibrosarcoma cell line in the rat (Günthert, U. et al., 1991, loc. cit.). Whereas the smallest CD44-isoform, the standard form sCD44, is expressed ubiquitously in a number of different tissues, including epithelial cells, certain splice variants of CD44 (vCD44) are expressed only on a subgroup of epithelial cells. The CD44-isoforms are produced by alternative splicing so that the sequences of 10 exons (v1-v10) in sCD44 are excised completely, but may occur in various combinations in the larger variants (Screaton, G. R. et al., 1992, loc. cit.; Heider, K.-H. et al., J. Cell. Biol. 120:227-233, 1993; Hofmann, M. et al., Cancer Res. 51:5292-5297, 1991). The variants differ in that different amino acid sequences are inserted at a certain point of the extracellular part of the protein. Such variants can be detected in different human tumour cells and in human tumour tissue. Thus, the expression of CD44-variants in the course of colorectal carcinogenesis has recently been investigated (Heider,K.-H. et al., 1993, loc. cit.). The expression of CD44-variants does not occur in normal human tissue (e.g. colon epithelium) and only slight expression can be detected in the proliferating cells of the crypts. At later stages of tumour progression, e.g. in adenocarcinomas, all malignant degenerations express variants of CD44. Moreover, the expression of CD44-splice variants was recently shown in activated lymphocytes and in non-Hodgkin's lymphomas (Koopman, G. et al., J. Exp.Med. 177:897-904, 1993).

The publication by Tölg, C. et al., Nucleic Acids Res. 21(5):1225-1229, 1993, describes how CD44 occurs in various isoforms. It discloses the amino acid sequences of ten different exons v1 to v10 in the mouse, rat and human:

The amino acid sequence of exon v4 of human vCD44 runs as follows (single letter code):

ISTTPRAFDHTKQNQDWTQWNPSHSNPEVLLLQTTTRMT

The amino acid sequence of exon v5 of human vCD44 reads as follows (single letter code):

DVDRNGTTAYEGNWNPEAHPPLIHHEHHEEEETPHSTST

The amino acid sequence of exon v6 of human vCD44 reads as follows (single letter code):

QATPSSTTEETATQKEQWFGNRWHEGYRQTPREDSHSTTGTA

The amino acid sequence of exon v9 of human vCD44 reads as follows (single letter code):

QQSNSQSFSTSHEGLEEDKDHPTTSTLTSS

WO 91/17248 describes the use of anti-vCD44 antibodies for the treatment and diagnosis of tumours. WO 95/00851 relates to the use of antibodies directed against variant exons of CD44 for the diagnosis and analysis of tumours. WO 95/04547 describes the use of such antibodies for immunotherapeutic and immunoscintigraphic purposes which are directed particularly against the variant exon v5. WO 95/33771 describes antibodies against variant exon v6 of CD44. EP-A 0 538 754 describes the use of antibodies for immunosuppression directed against variant CD44 (vCD44).

The aim of the present invention was to provide means for treating certain tumours and for suppressing immune responses and developing processes for the preparation of such means.

This aim has been achieved with the present invention within the scope of the specification and claims by using, on the one hand, antibodies against the constant part of CD44 (standard form, sCD44) and/or against the variant forms of CD44 (vCD44) in order to treat malignant diseases associated with the degeneration and activation of Langerhans cells (LC) and/or dendritic cells (DC), and on the other hand using antibodies against the constant part of CD44 (sCD44) in order to suppress unwanted or excessive immune reactions and treat or positively influence diseases and events caused by such reactions.

According to another aspect of the present invention, dendritic cells (DC) prepared by certain methods are used to trigger or initiate immune reactions in vivo in order to carry out an adoptive immunotherapy, for example.

In connection with this, the present invention also relates to an ex vivo cultivation process for preparing dendritic cells which preferably migrate into peripheral lymph nodes.

A cultivation process of this kind also solves the problem of giving dendritic cells the ability to migrate in controlled manner into the peripheral lymph nodes, adhere therein in the T-cell areas and initiate a T-cell-mediated immune reaction. This is of major importance for the use of dendritic cells of this kind for immunotherapeutic purposes.

Preferred antibodies for the above-mentioned uses according to the invention are those which react with epitopes of the N-terminal portion of the constant parts of CD44 (sCD44) or which recognise the epitopes which are coded by the variant exons v4, v5, v6 or v9, of which antibodies against v6 are particularly preferred. The term N-terminal portion of the constant part of CD44 is intended to refer to that part of the protein which is coded by the constant exons 1 to 5 of the CD44-gene according to the publication by Screaton et al. (Screaton G R et al., Proc. Natl. Acad. Sci. USA 89: 12160-12164, 1992). Particularly preferred are antibodies which bind specifically to an epitope coded by exon 2, 3, 4 and/or 5.

Examples include the monoclonal antibodies BU-52 (The Binding Site, Birmingham, England, Cat.No. MC114; Messadi, D. V and Bertolami, C. N., Am J. Pathol. 142:1041-1049, 1993; Spring, F. A. et al., In: Leucocyte typing V, white cell differentiation antigens, Schlossman, S. F., Boumsell, L., Gilks, W., Harlan, J. M., Kishimoto, T., Morimoto C., Ritz, J. and Shaw, S. (Ed.), Oxford, New York, Tokyo:Oxford University Press, 1995), SFF-2 (Boehringer Ingelheim Bioproducts, Cat.No. BMS113) or MEM-85 (Boehringer Ingelheim Bioproducts, Cat.No. BMS5033Fl.01; Bazil, V. et al., Folia Biologica 35(5):289-297, 1989; Spring, F. A. et al., In: Leucocyte typing V, white cell differentiation antigens, Schlossman, S. F., Boumsell, L., Gilks, W., Harlan, J. M., Kishimoto, T., Morimoto C., Ritz, J. and Shaw, S. (Ed.), Oxford, New York, Tokyo:Oxford University Press, 1995) which recognise epitopes of the N-terminal portion of the constant part of CD44.

A preferred antibody against the N-terminal portion of the constant part of CD44 (sCD44) is the monoclonal antibody SFF-2 which is secreted by a hybridoma cell line which was deposited on 16.04.1997 under deposit number DSM ACC2305 at the DSM-Deutsche Sammlung fur Mikroorganismen und Zell-kulturen GmbH, Mascheroder Weg 1b, D-38124 Braunschweig, Germany, or derivatives of this antibody.

One aspect of the present invention is therefore the use of anti-sCD44 and/or anti-vDC44 antibodies for producing a preparation for the prophylactic and therapeutic treatment of malignant diseases which are associated with a degeneration, activation or massive multiplication of Langerhans cells (LC) and/or dendritic cells (DC). Cf. for example Langerhans-cell-histiocytoses, histiocytosis X, Abt-Letterer-Siewe syndrome, eosinophilic granuloma (Simon, J. C. et al., Hautarzt 43:241-249, 1992).

According to another aspect the present invention relates to the use of anti-sCD44 antibodies which recognise N-terminal epitopes of sCD44 or sections thereof, for producing a preparation for the prophylactic and therapeutic treatment of malignant diseases which are associated with a degeneration, activation or massive multiplication of Langerhans cells (LC) and/or dendritic cells (DC).

According to yet another aspect the present invention relates to the above-mentioned uses of anti-sCD44 antibodies, where the antibodies are monoclonal antibodies or fragments or derivatives thereof.

In an additional aspect the present invention relates to the use of antibodies directed against epitopes which are coded by variant exons of vCD44 or parts thereof, for producing a preparation for the prophylactic and therapeutic treatment of malignant diseases which are associated with the degeneration, activation or massive replication of Langerhans cells (LC) and/or dendritic cells (DC).

In one particular embodiment the present invention relates to the use of antibodies directed against epitopes which are coded by the variant exons v4, v5, v6 and/or v9 or parts thereof, for producing a preparation for the prophylactic and therapeutic treatment of malignant diseases which are associated with the degeneration, activation or massive multiplication of Langerhans cells (LC) and/or dendritic cells (DC).

In particular, the invention relates to the above-mentioned use of antibodies which are capable of reacting with the following amino acid sequences or parts thereof:

ISTTPRAFDHTKQNQDWTQWNPSHSNPEVLLLQTTTRMT
DVDRNGTTAYEGNWNPEAHPPLIHHEHHEEEETPHSTST
QATPSSTTEETATQKEQWFGNRWHEGYRQTPREDSHSTTGTA
QQSNSQSFSTSHEGLEEDKDHPTTSTLTSS

In another particular embodiment, monoclonal antibodies and fragments and derivatives thereof are used for the purposes according to the invention as described above.

In a most particular embodiment the invention relates to the use of the antibody VFF-18, which is secreted by a hybridoma cell line, which was deposited on 7.6.1994 under deposit number DSM ACC2174 at the DSM-Deutsche Sammlung für Mikroorganismen und Zellkulturen GmbH, Mascheroder Weg 1b, D-38124 Braunschweig, Germany, (WO 95/33771), or derivatives of this antibody.

An additional aspect of the present invention relates to the use of anti-sCD44 antibodies for producing a preparation for suppressing or alleviating unwanted or excessive immune reactions for the treatment or prophylaxis of diseases caused thereby or in order to have a beneficial effect on events connected therewith.

Consequently, the use of anti-sCD44 antibodies covers all those diseases and conditions of a mammalian body which are based on an immunoregulatory disorder or an unwanted or excessive immune reaction, such as allergic illnesses, particularly allergies of the delayed type, rejections of skin or organ transplants, autoimmune diseases, diseases of the rheumatic type, multiple sclerosis, psoriasis or atopical dermatitis.

The anti-sCD44 antibodies described are suitable for this purpose, both in a prophylactic capacity and for therapeutic treatments.

In another aspect the invention covers monoclonal anti-sCD44 antibodies for the above-mentioned uses for influencing immune reactions in terms of prophylactic and therapeutic treatments, the antibodies being monoclonal antibodies or fragments or derivatives thereof.

One particular embodiment relates to the above-mentioned uses for influencing immune reactions of the antibody VFF-18 directed against the epitope coded by v6, or parts thereof, and those antibodies which are capable of reacting with the epitope recognised by VFF-18 or parts thereof.

According to another aspect the present invention relates to the production of dendritic cells and their use for triggering or initiating an immune reaction in vivo for immunotherapy, particularly adoptive immunotherapy, e.g. the adoptive immunotherapy of tumours or virus diseases.

It has been found that in the course of migrating into the lymph nodes LC and DC modulate their expression of CD44 isoforms. In particular, epitopes in the N-terminal constant part of CD44 and epitopes coded by the variant exons v4, v5, v6 and v9 are regulated. It has also been found that the modulation of CD44-isoforms in stages is of great importance to the immune function of the LC and DC. In other words, it has been found according to the invention that the activation and migration of LC out of the epidermis can be totally prevented by antibodies, particularly monoclonal antibodies, against N-terminal epitopes in the constant part of CD44 (sCD44). Moreover, it has surprisingly been found that the specific accumulation of LC and DC in the T-cell areas of the peripheral lymph nodes can be totally inhibited by means of antibodies which recognise epitopes on variant exons, e.g. v6 or CD44v6 (for example VFF18, disclosed for example in WO 95/33771). However, it has also been found that antibodies directed against an N-terminal epitope in the constant part of CD44 (sCD44) also blocked the adhesion, albeit to a lesser extent than the reaction of antibodies against variant exons.

It has been confirmed in a mouse model that the systemic administration of antibodies, particularly monoclonal antibodies, against variant epitopes of CD44, such as CD44v6, inhibit the ability of LC and DC to trigger an allergy to a hapten, such as 2,4-dinitrofluorobenzene. These antibodies were effective even when the allergy was already in existence. Surprisingly, it was found that in addition to the CD44v6-specific monoclonal antibodies, antibodies against N-terminal epitopes of the constant part of CD44 (sCD44) were also effective here. Thus, the use of antibodies directed against sCD44 opens up the possibility of both a prophylactic and a therapeutic treatment of immune processes in vivo.

In a developed cell cultivation process it was possible to produce DCs which migrate into peripheral lymph nodes and preferentially accumulate in the T-cell areas. This process is based, according to the invention, on the fact that monocytes are isolated from the peripheral blood of healthy blood donors or from patients with malignant tumours, e.g. melanomas, or from the bone marrow, and the cells thus obtained are cultivated for some days in a suitable culture medium, e.g. RPMI 1640, supplemented with serum, antibiotics, non-essential amino acids, a buffer, e.g. a buffer which is active in the range from pH 6.0 to pH 8.5, particularly an organic buffer, and at least one cytokine, and are then isolated.

In a preferred embodiment, the process is characterised in that the monocytes isolated as described above are cultivated for some days in a culture medium consisting of RPMI 1640, foetal calf serum, penicillin/streptomycin, a buffer consisting of an N-substituted aminosulphonic acid, such as Hepes [4-(2-hydroxyethyl)-1-piperazine ethanesulphonic acid], non-essential amino acids, L-glutamine, GM-CSF (granulocyte/macrophage colony-stimulating factor, described for example in WO86/03225 or by Cantrell, M. A. et al., Proc. Natl. Acad. Sci. USA 82:6250-6254, 1985), and then isolated.

In a most particular embodiment given by way of example, the process is characterised in that the monocytes isolated as described above are cultivated for 8 days in a culture medium consisting of RPMI 1640, 10% FCS (foetal calf serum), 45 μg of penicillin/streptomycin, 25 mM Hepes, 1 mM non-essential amino acids, 2 mM L-glutamine, 50 ng/ml human GM-CSF, preferably recombinant human GM-CSF, and 1000 U/ml of interleukin-4 (IL-4) and then isolated.

Surprisingly, it was found that after the cultivation process according to the invention the culture consisted of >90% dendritic cells which had the same CD44-isoform pattern as LC or DC which had migrated into lymph nodes in vivo. The DC cultivated according to the invention bind, like activated LC, into the paracortical T-cell areas of lymph nodes. This binding was blocked by antibodies which epitopes coded by the variant exons v4, v5, v6 or v9, for example by the monoclonal antibody VFF18, or which react with antibodies against the constant part of CD44 (standard form, sCD44, preferably with antibodies against epitopes of the N-terminal portion of the constant part of CD44 (sCD44).

By means of these dendritic cells produced ex vivo, an immunotherapy, particularly adoptive immunotherapy such as the adoptive immunotherapy of tumours or virus diseases, can advantageously be carried out in vitro. Such immunotherapy is consequently based on influencing the migration characteristics of the DC prepared from the blood or bone marrow according to the invention in such a way that the dendritic cells according to the invention migrate into the peripheral lymph nodes where they initiate the desired immune reactions. These cells can advantageously enhance the immunogenicity of DC when used in adoptive immunotherapy of inflammatory, infectious, proliferative or hyperproliferative diseases, e.g. virus or tumour diseases.

Since both the DNA and the amino acid sequences of sCD44 and vCD44 are known, the skilled person can use any desired variant vCD44 glycoprotein or variant forms thereof (vCD44) of animal or human origin and the nucleic acids coding for them (DNAs and KNAs) in order to produce and use any desired antibodies directed against sCD44 or variant forms thereof (vCD44), especially monoclonal antibodies, fragments and derivatives thereof.

The terms sCD44 or vCD44 used as a basis for the present invention refer to proteins or parts or epitopes thereof which are coded by RNA, DNA or transcripts coding for sCD44 or vCD44, including those which are altered by mutations, e.g. by deletions, insertions, substitutions, inversions, transitions and transversions and those which hybridise with the DNA sequences described in the prior art, for example in Tölg, C. et al., Nucleic Acids Res. 21(5) :1225-1229, 1993, or in Srceaton, G. R. et al., Proc. Natl. Acad.-Sci. USA 89:12160-12164, 1992, under the known conventional conditions. It is unimportant whether these nucleic acids are prepared and isolated conventionally by means of cell cultures or by DNA-recombination, by synthetic or semisynthetic methods.

The terms sCD44 and vCD44 within the scope of the present invention denote all those glycoproteins obtained from animals or humans, irrespective of their production or isolation by means of conventional cell cultures or by DNA recombination or by synthetic or semi-synthetic processes.

The term antibodies refers to mono- or polyvalent antibodies and poly- and monoclonal antibodies and also those which are fragments thereof and derivatives thereof, including the F(ab′)₂, Fab′ and Fab fragments, but also chimeric antibodies or hybrid antibodies having at least two antigen or epitope binding sites (such as quadromas, triomas), interspecies hybrid antibodies, antiidiotypic antibodies and those which are chemically modified and can be regarded as derivatives of these antibodies and which can be obtained either by the known conventional methods of antibody recovery or by DNA-recombination (such as diabodys), via hybridoma techniques or antibody engineering or synthetically or semi-synthetically by methods known per se and which are capable of binding to epitopes of sCD44 or vCD44. Humanised antibodies may be prepared for example by CDR-grafting (EP 0239400). Framework regions can also be modified (EP 0519596; WO 9007861). Nowadays it is possible to humanise antibodies using methods such as PCR (cf. for example EP 0368684; EP 0438310; WO 9207075) or computer-modelling (see for example WO 9222653). Reference is hereby made to the extensive literature which has appeared since Köhler, G. & Milstein, C., Nature 256:495-497, 1975, which is known to those skilled in the art.

With regard to the production of polyclonal antibodies against epitopes of sCD44 and vCD44 there are a number of methods available. For example, various animals can be immunised for this purpose in a manner known per se by injection with sCD44 or vCD44, which may be of natural origin or prepared by DNA-recombination or synthetically, or fragments thereof, and the desired polyclonal antibodies are obtained from the resulting sera by known methods and the purified. Alternatively, intact cells may also be used. Various adjuvants for increasing the immune response to the administration of sCD44- or vCD44-dose may also be used, depending on the animal chosen for the immunisation, such as Freund's adjuvant, mineral gels such as aluminium hydroxide, surface active substances such as polyanions, peptides, oil emulsions, haemocyanins, dinitrophenol or lysolecithin.

The monoclonal antibodies against an epitope of: sCD44 or an epitope of vCD44, which are preferred for the purposes of the invention, may be obtained by any desired technique which is available for the production of antibodies by the cultivation of cell lines. Such known techniques include, for example the methods described by Köhler, G. & Milstein, C., 1975, loc. cit., or Taggart & Samiloff, Science 219,:1228-1230, 1983, using hybridoma cells, or those with human B cell hybridomas (Kozbor et al. Immunology Today 4,:72-79, 1983). Chimeric antibodies against sCD44 or vCD44 or parts thereof may be made up, for example, of a mouse antigen binding domain and human constant regions (Morrison, et al., Proc. Natl. Acad. Sci. USA 81,:6851-6855, 1984; Takeda, et al., Nature 314,-452-454, 1985).

The antibodies may be purified by known methods, e.g. by immunoabsorption- or immunoaffinity chromatography, by HPLC (High Performance Liquid Chromatography) or combinations thereof. Antibody fragments which contain the idiotype of the molecule may also be prepared by known methods. For example, F(ab′)₂ fragments may be obtained by pepsin digestion of the total poly- or monoclonal antibody. Fab′ fragments may be obtained by reducing the disulphide bridges of the relevant F(ab′)₂ fragment, for example, and Fab fragments may be prepared for example by treating the antibody molecules with papain and a reducing agent.

Any known method may be used to identify and select antibodies, fragments or derivatives thereof which react with an epitope of sCD44 or vCD44. For example, by the fact that the antibodies in question are detectable after suitable labelling when they have bound to isolated or purified sCD44 or vCD44 or parts thereof or by immunoprecipitation of the sCD44 or vCD44 purified by means of polyacrylamide gels for example, or by the fact that antibodies against sCD44 or vCD44 compete with other anti-sCD44 or anti-vCD44 antibodies to bind to sCD44 or vCD44 or parts thereof.

However, the invention also includes the use of hybridoma cell lines for the production of the antibody-containing preparations described in the present invention for the purposes on which the invention is based.

For further information regarding the general use of monoclonal antibodies for immunosuppression and in autoimmune diseases, of hybrid antibodies for therapeutic purposes and antibodies produced by DNA recombination, reference is made, for example, to Progress in Allergy Vol. 45, “Monoclonal AntibodyTherapy”, 1988 and the study by Seaman, W. E. et al., Ann. Rev. Med. 39,:231-241, 1988.

The CD44 glycoprotein (standard form, sCD44) including its isoforms and variants (vCD44, v1 to v10) are fully described in the literature, both for animals (e.g. rat, mouse) and for humans, in terms of its substance parameters (DNA- and amino acid sequences, location within the complete gene coding for CD44) and its production, so that by means of this disclosure it is possible for the skilled person to produce any antibodies or monoclonal bodies or parts and derivatives thereof according to the above definitions for each epitope located on this protein and use them according to the invention, so that their use is not restricted to certain specific antibodies or the hybrid cell lines which produce them.

In general, preparations with antibodies of this kind can be used in the tumoral diseases and immune processes in animals and humans as described in this specification which comprise prevention or prophylaxis, or therapeutic treatment.

On the basis of their immunosuppressant activity established according to the invention, the designated antibodies or the preparations containing them are suitable for the prevention and treatment of diseases and conditions which require a temporary or permanent reduction or suppression of immune response.

In particular, their use in vivo includes the treatment and prophylaxis of undesirable or excessive immune responses harmful to the mammalian organism in question, by preventing LC and DC from docking at T-cell areas of peripheral lymph nodes. For example, in order to prevent or treat autoimmune diseases such as diseases of the rheumatic type, multiple sclerosis, psoriasis, atopical dermatitis, or to prevent the rejection of transplanted tissues or organs such as e.g. kidneys, hearts, lungs, bone marrow, spleen, skin or cornea, in undesirable reactions during or after transfusions, allergic diseases, e.g. those which affect the gastro-intestinal tract and may have an inflammatory action therein, or inflammatory, proliferative and hyperproliferative diseases and cutaneous manifestations of immunologically caused diseases such eczematous dermatitis, urticaria, vasculitis and sclerodermy.

Depending on the nature and cause of the disease or disorder to be treated or the condition to be influenced in an animal or human body it may be desirable to administer the antibody preparation systemically, locally or topically to the tissue or organ in question. A systemic approach is desirable, for example, if different organs or organ systems require treatment, as in systemic autoimmune diseases or allergies or in the transplantation of foreign, fairly large organs or tissue or in tumours which are difficult to locate. By contrast, a local effect would be considered if only local manifestations of a neoplastic or immunological occurrence had to be treated, e.g. in local tumour events, small-area transplants of skin and corneas or in local immunological reactions, e.g. of the skin, such as local dermatitis.

The antibodies in question may be administered by any enteral or parenteral route known to those skilled in the art. For systemic administration, the intravenous, intravascular, intramuscular, intraarterial, intraperitoneal, oral or intrathecal route may be used, for example. Rather more local administration can be achieved subcutaneously, intracutaneously, intracardially, intralobal, intramedullary, intrapulmonary route or into or onto the tissue which is to be treated (connective tissue, bone, muscle, nerve, epithelial or bone tissue). Depending on the duration and potency of the immunosuppressant activity desired, the antibody preparations may be administered once or repeatedly, possibly intermittently, per day for a period of several days, weeks or months and in different doses.

An antibody preparation suitable for the above-mentioned purposes may be prepared using the injectable, physiologically acceptable solutions known to the skilled person in sterile form. A ready to use solution for parenteral injection or infusion may be prepared using the known aqueous isotonic solutions such as saline or a corresponding plasma protein solution without gamma globulin. However, the preparation may also be in a lyophilised or dry form which can be reconstituted under sterile conditions immediately before use with one of the known injectable solutions, e.g. as a kit of parts. The final production of an antibody preparation to be used according to the invention for injection, infusion or perfusion is carried out by mixing antibodies purified by known methods according to the definitions given hereinbefore with one of the above-mentioned physiologically acceptable solutions, which may optionally be supplemented with known carriers or excipients (e.g. serum albumin, dextrose, sodium bisulphite, EDTA).

The amount of antibody to be administered depends on the nature and seriousness of the illness or disorder to be treated or the condition to be influenced and the patient in question, be they animal or human. However, the starting point is a dosage of from 1 to 1000 mg, preferably 5-200 mg of the antibody per single dose, as is conventional for other antibodies or monoclonal antibodies, whilst the quantity to be administered may be from 0.01 to 20 mg/day and 0.1 to 100 mg/kg of body weight/day even over a long period (days, weeks, months) in order to achieve the desired effects, depending on how intensively and over what length of time a prophylactic or therapeutic effect is to be achieved.

In order to investigate whether Langerhans cells express CD44 and whether this expression is altered during LC activation, freshly isolated LC from human epidermis was placed in a culture. This process imitates LC activation by antigen (Schuler, G & Steinman, R. M., J. Exp. Med .161:526-546,1986). Epidermal cell suspensions enriched with LC were subjected to a three-colour FACS analysis [“fluorescence activated cell sorting”] either immediately after isolation from the skin or after 48 and 72 hours of the epidermal total cell culture. The cells were stained with monoclonal antibodies (mAbs) against HLA-DR in order to identify LC and with mabs against CD44 epitopes. Freshly isolated HLA-DR⁺LC(fLC) expressed an N-terminal epitope of CD44 (known as “pan CD44”) and an epitope which was formed jointly by CD44 exons v7 and v8 (FIG. 1a). Epitopes which are coded by exons v5 and v6 were only weakly expressed, whereas epitopes coded by v4, v9 and v10 were undetectable (FIG. 1a). LC which had been cultivated for 48 and 72 hours carried increased levels (2-fold) of N-terminal epitopes. The epitopes of v4, v5, v6 and v9 were also downregulated (FIG. 1a). By contrast, CFD44v7/8 was lost during cultivation (FIG. 1a). No CD44 v10 epitope could be detected. From this it can concluded that the LC activation is accompanied by increased synthesis of CD44 and a change in either the accessibility for the epitope or the epitope splicing.

LCs belong to the family of the dendritic cells (Steinman, R. M., Annu. Rev. Immunol.9:271-296, 1991). In order to determine whether DCs of other origins express CD44 epitopes which resemble those of LC, DCs were prepared from peripheral blood by cultivation in a cytokine-cocktail (Sallusto, F. & Lanzavecchia, A., J. Exp. Med. 179:1109-1118, 1994). FACS analysis of CDla⁺ (DC marker) cells showed up CD44 epitope patterns which were identical to those of cultivated LCs (FIG. 1b). A very similar pattern of CD44 expression was also produced in cultivated LCs obtained from the skin of BL/6 mice (FIG. 1c).

In order to determine at what stage LC undergoes a changed expression of CD44 epitopes during cultivation, a skin explant culture (Larsen, C. P. et al., J. Exp. Med. 172:1483-1493, 1990) was used into which LC actively migrates from the epidermis into the dermis. Complete whole thickness punch biopsies from human skin were cultivated for between 0-72 hours. In frozen sections, LCs were identified both under a light microscope with Lag mAb against Birbeck Granula (cytoplasmic organelles specific to human epidermal LCs (Kashihara, M. et al., J. Invest. Dermatol. 87:601-607, 1986)) and by transmission electron microscopy. In freshly isolated skin, virtually all the Lag⁺-cells were located in the suprabasal layers of the epidermis, with very few detectable Lag⁺-cells in the dermis, whereas after 72 hours cultivation almost half the LCs had left the epidermis. Just 2 hours after the start of cultivation, epidermal LCs began to migrate and after 12 hours it was established that they had penetrated the basal membrane of the epidermo-dermal junction points. After 24 hours the LCs had accumulated in the dermis in rope-like structures inside lymphatic vessels, which was identified by their characteristic ultrastructural features of a “single layer” of endothelial cells with external nuclei under the electron microscope.

By immunohistochemical double labelling with mAb Lag (FITC, green) and CD44 specific antibodies (Cy3, red), the expression of CD44 on LC was monitored during migration. Double staining showed up as yellow. Keratinocytes were stained red as they carry different CD44 epitopes (CD44v2-v10) (Hofmann, M. et al., Cancer Res. 53:1516-1521, 1993; Hudson, D. L. et al., J. Cell. Science 108(Pt 5):1959-1970, 1995), but not the Lag epitopes. Most of the intra-epidermal LCs expressed both N-terminal epitopes and also the epitopes coded by v7/8, whereas only a few (≦5%) thereof exhibited staining with antibodies against epitopes coded by exons v5, v6 or v9 (FIGS. 2a, b, c, e, g). By contrast, the majority of the LCs which migrated into the dermis (87.5-100%) expressed v5, v6 and v9 epitopes (FIGS. 2d, f, h). Thus, the change in the epitope presentation obviously takes place during the transition from epidermis to dermis. In order to confirm these observations, (“split thickness skin”) was floated on culture medium and the LCs were allowed to migrate into the medium. After 48 hours cultivation the LCs were collected. These LCs carried epitopes of the CD44 N-terminus, v5, v6 and v9, but not v7/8 (FIG. 2 l, m). Thus, the CD44 phenotype of LCs which migrated totally out of the skin exactly coincided with those of the LCs or DCs activated in vitro.

In order to monitor the LC migration further, LCs were identified in frozen sections of axilliary lymph nodes; these had migrated via lymphatic pathways to the regional lymph nodes. In the paracortical T-cell zones of lymph nodes, Lag⁺ cells were found and the majority expressed epitopes of exons v4, v5, v6 and v9 (FIG. 2k) in addition to the N-terminus of CD44 (FIG. 2i), but not the v7/8 epitope. From this it can be concluded that the CD44 expression pattern obtained during emigration from the epidermis remains until the LCs have reached the lymph nodes.

Anti-CD44 antibodies were used to determine the functional significance of the CD44 protein expression during the early stages of LC activation, during the adhesion of LCs and DCs to lymph nodes and during the antigen presentation by LCs.

With regard to the investigation of the CD44 function during early LC activation, both anti-CD44 N-terminal antibodies, which on the one hand block the hyaluronic acid (HA) binding (for example MEM-85, Bennett, K. L. et al., J. Cell. Biol. 128:687-698, 1995) and on the other hand which do not block HA binding, as well as v5, v6 or v9 specific mAbs were added to the medium with the floating (“split thickness keratome skin”) and the incidence of LC in the medium was counted by FACS. LC was retained in the epidermis by both N-terminal specific antibodies (60% to 80% inhibition) but not by the exon specific mAbs (FIG. 3). Other antibodies which recognise the N-terminus also exhibited inhibition, whereas antibodies against the v7/v8 epitope did not have an inhibitory effect. These results show that CD44 is involved in a very early stage of LC activation;. When LCs are first activated and express epitopes coded by exons v5, v6 and v9, antibodies which recognise these epitopes cannot interfere with the migration of LCs out of the epidermis.

In order to determine the role of CD44 during the adhesion of activated LCs and DCs at paracortical T cell areas in sections of lymph nodes, various anti-CD44 antibodies were used to block this adhesion (FIG. 4) . MACS® (Microsphere Activated Cell Sorting)-purified fresh LCs, LCs and DCs from blood cultivated for 48 hours (and activated) were used for a lymph noden frozen section binding assay (Butcher, E. C., et al., J,. Immunol. 123:1996-2003, 1979). LCs or DCs were identified by counter-staining with a suitable antibody (such as CDla mAb or OKT-6, Ortho, Neckargemünd) and the specific adhesion to lymph nodes was determined by microscopy. Fresh LCs or immature DCs formed by peripheral blood showed only weak binding to lymph node frozen sections (FIG. 4a). Cultivated LCs and cytokine-activated DCs adhered specifically and with greater efficiency to the paracortical T-cell areas but not to the central follicular B-cell areas (FIG. 4b, c). Pre-incubation with the CD44v6 specific VFF18 mAb significantly inhibited the binding of cultivated LCs (up to 66%) and DCs (up to 49%) to the T-cell zones (FIGS. 4d, e). An antibody directed against the N-terminal epitope in the constant part of CD44 (such as MEM-85, Boehringer Ingelheim Bioproducts, Cat.No. BMS5033Fl.0l; Bazil, V. et al., Folia Biologica 35(5) :289-297, 1989; Spring, F. A. et al., In: Leucocyte typing V, white cell differentiation antigens, Schlossman, S. F., Boumsell, L., Gilks, W., Harlan, J. M., Kishimoto, T., Morimoto C., Ritz, J. and Shaw, S. (Ed.), Oxford, New York, Tokyo:Oxford University Press, 1995) also inhibited binding, although less efficiently. Other mAbs which were directed against other v exon epitopes or against ICAM-1 showed no effect. The data obtained with VFF18 show that CD44 mediates the binding LC and DC to an unidentified partner in the T-cell zone of the lymph nodes. This effect, particularly of MEM-85, indicates a binding function to hyaluronic acid (which is presumably not restricted to the T-cell zones) or, which is more probable, to the same partner which is acted on VFF18.

The crucial test for LC function concerns its role in vivo in the presentation of an antigen which has come together with T-cells in the skin. For this purpose, mice were epicutaneously sensitised with DNFB (dinitrofluorobenzene) which, after stimulation or (“challenge”) with the same hapten in the skin of the ear results on day 6 in a massive DTH (“Delayed Type Hypersensitivity”) reaction measured by means of the swelling of the ear (FIG. 5). Anti-CD44 mAbs were injected i.p. on days −1, 0 and +1 with respect to the administration of the hapten (in order to test for interference with the sensitisation phase of DTH which requires LC migration from the epidermis to the lymph nodes and hapten presentation (Austyn, J. M., J. Exp. Med. 183:1287-1292, 1996)) and on days 5, 6 and 7 (in order to test for interference with the stimulation or (“challenge”) phase which requires extravasation of leucocytes (Camp, R. L. et al., J. Exp. Med. 178:497-507, 1993). The challenge dose of the hapten was administered on day 6. Whereas antibodies against the N-terminus of CD44 and against the v6 epitope inhibited the extravasation phase of DTH, only the v4 and v6 specific antibodies greatly inhibited the sensitisation (FIG. 5). From this, it can be concluded that CD44 variants play an essential part in LC function, either during the migration to the lymph nodes or during the interaction with T-cells. The inhibition of the extravasation phase of DTH by N-terminal CD44 antibodies is known (Camp, R. L. et al., 1993, loc. cit.). The inability of N-terminal-specific anti-CD44 antibodies to inhibit the LC activation in the epidermis in vivo (FIG. 5), whereas they are capable of doing it in vitro (FIG. 3), is presumably down to the basal membrane barriers which prevent the effective entry of antibodies into the epidermis after systemic application (Camp, R. L. et al., 1993, loc. cit.).

The interference of anti-CD44v6 antibodies with the LC function at least partly explains their effect on the primary immune response (Arch, R., et al., Science 257:682-685, 1992; Moll, J. et al., J. Immunol. 156:2085-2094, 1995). The parallels found between the LC behaviour and the lymphatic spread of metastasising tumour cells and the inhibition of both by anti-CD44v6 antibodies demonstrates that CD44 plays the same role in both processes. Accordingly, it must be assumed that the selection for molecular function and the imitation of the molecular functions of CD44 on LC and DC take place during the tumour progression.

### DETAILS OF THE FIGURES

FIG. 1: Change in the CD44 epitope expression during in vitro cultivation of epidermal LCs and blood DCs. (a) Freshly isolated human LC and after 48 and 72 hours' cultivation and (c) mouse BL/6 skin-LC after 72 hours' cultivation were stained with mAbs against HLA-DR or Ia^{b}, 7-AAD and mabs against CD44 epitopes. The average fluorescence intensity of HLA-DR⁺ cells was determined by means of an FACScan. Representative results for 6 experiments with cells from different donors. Low expression of CD44v epitopes on fLC was not caused by the trypsinisation used during the isolation process, since the identical trypsinisation of cultivated LC did not significantly influence their CD44v expression. (b) Corresponding analysis of DC generated from human peripheral blood monocytes, selective determination of CDla positive cells. Representative results for 4 experiments with cells from different donors.

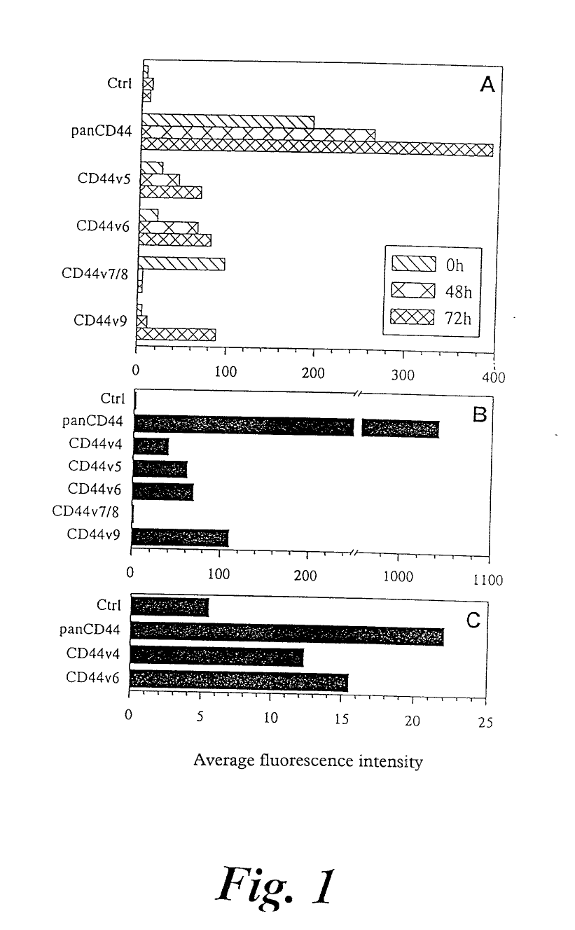

FIG. 2: LCs migrating from the epidermis into dermis and lymph nodes exhibit the same CD44 expression pattern as LC/DC activated in vitro. Frozen section of skin cultivated for 12 hours were stained with FITC-conjugated mAK goat-anti-mouse Lag (recognises Birbeck Granula, specific cytoplasmatic organelles of LZ, Kashihara, M. et al., J. Invest. Dermatol.87:601-607, 1986) mAbs in order to detect LCs (green), and with Cy3-conjugated goat-anti-mouse CD44 mAbs (red). Double-positive cells appear yellow-orange. The bar indicates 31 μm. The same magnification from A to K. (a) Staining with Lag and anti pan CD44 antibody (recognise standard part of the CD44 molecule). Lag⁺ cells inside the epidermis (*) and those which had migrated into the dermis (**) were stained double positive for Lag and pan CD44 (yellow). Keratinocytes expressed pan CD44 but not Lag (red). Lag⁺ intra-epidermal LC also expressed the epitope formed by exons v7/v8 (b) but at very low levels expressed the epitopes of CD44 exon v5 (c), v6 (e) and v9 (g). Lag⁺ cells which had migrated into the dermis expressed CD44v5 (d), v6 (f) and v9 (h). (i) Pan CD44 staining of Lag⁺ cells (yellow) which are located in the paracortical T-cell areas of the axillary lymph nodes and drain the skin. T-cells also expressed CD44 but not Lag (red). (k) The exon v9 epitope is expressed in the majority (yellow) but not all Lag positive cells (arrow, green). (l, m) LC which had migrated out of (“split-thickness skin”) were selected after HLA-DR expression and analysed with the specified mAbs by FACS as in FIG. 1.

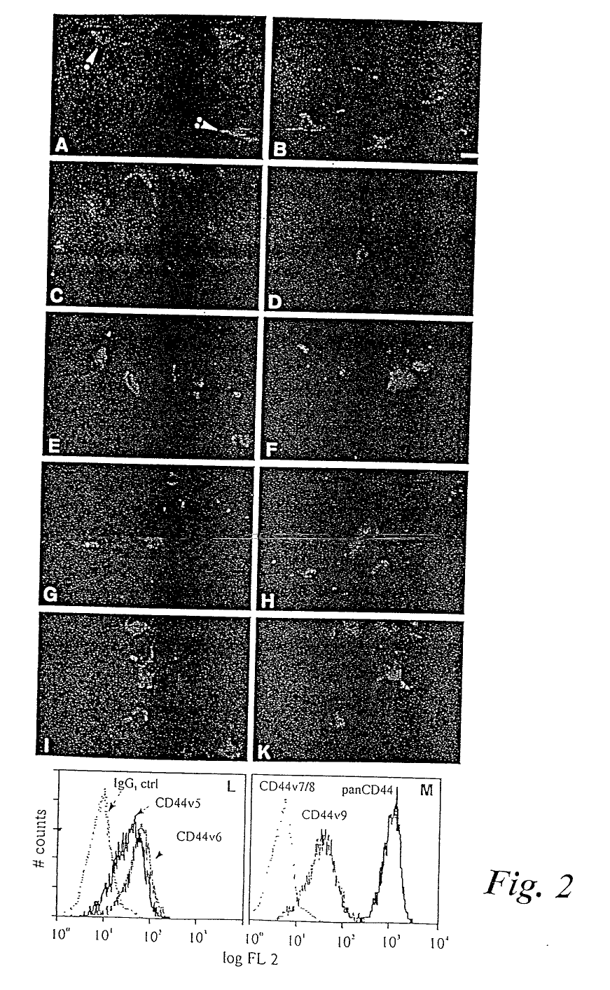

FIG. 3: Antibodies against the CD44 N-terminus prevent LC activation and/or migration in vitro. Antibodies were added to (“split-thickness skin cultures”) and the cells which had migrated into the medium out of the split-thickness skin were analysed by FACS. (a) Treatment with MEM-85 or control mAb. CD1⁺, live (propidium iodide-negative) LCs are circled. The fraction of these cells over the total cell count is indicated in % in the top corner of each graph. (b) Inhibition of LC emigration by variants anti-CD44 antibodies. The percentage inhibition was calculated from 5 independent experiments (+/- SD, * statistically significant at p<0.05, “one way ANOVA”, Dunnett's Test, SigmaStat, Jandel Scientific Software).

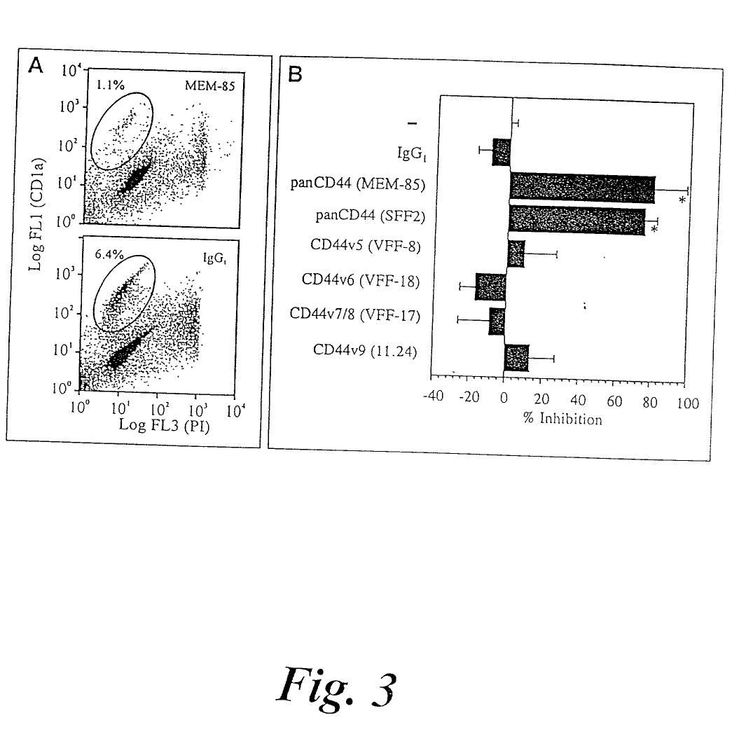

FIG. 4: The binding of LC or DC to paracortical lymph node areas is inhibited by antibodies against CD44. Fresh LC (a) purified with microbeads, LC cultivated for 48 hours (b, d) or cytokine-activated DC (c, e) were placed on frozen sections of lymph nodes (bar =31 μm). LC and DC adhered preferentially to the paracortical T-cell areas, the adhesion increasing as a result of activation by the culture, but not to the central B cell follicles. The binding was also determined in the presence of antibodies (d,c) (quantified in d and e, *=statistically significant at p<0.05, “one way ANOVA”, Dunnett's Test).

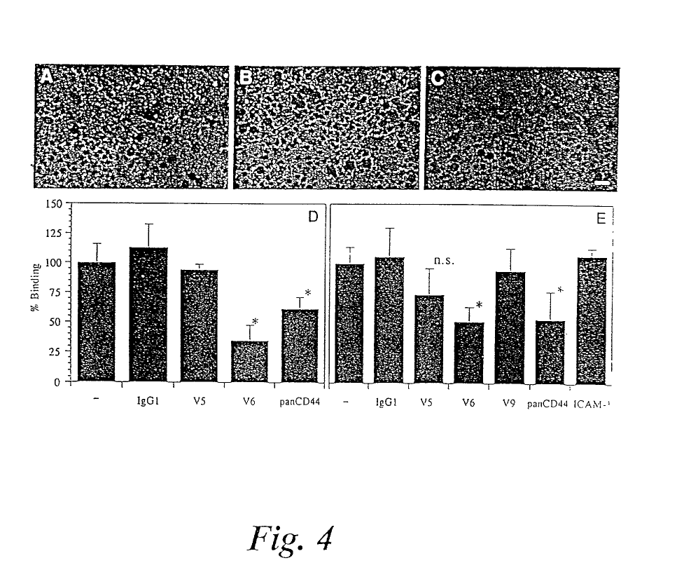

FIG. 5: Antibodies against CD44v epitopes inhibit the sensitisation phase of contact-hypersensitivity in vivo. The group 2 mice were treated with the specified mAbs i.p. both before and during the sensitisation phase and the group 3 mice were similarly treated before and during the “challenge phase”. Mice of group 1 were not treated with mabs. * statistically significant way “NOVA”, Dunnett's Test)

The Examples which follow are intended to illustrate the invention.

### EXAMPLE 1

Isolation of the Langerhans cells (LC) and the dendritic cells (DC)

Human epidermal cell suspensions were obtained by the method of Simon, J. C., et al., Exp. Dermatol. 4:155-161, 1995, by limited trypsinisation of human skin obtained in plastic surgery. Murine LC was taken from the skin of the rump of female C57/BL63 mice (Harlan-Olac, Great Britain) according to Simon, J. C., et al., J. Immunol. 146:485-491, 1991. LC were concentrated by density gradient centrifugation on Lymphoprep (Gibco) to 5-20%. Both fresh LC and also LC cultivated for 48 or 72 hours in supplemented RPMI (Simon, J. C., et al., Exp. Dermatol. 4:155-161, 1995; Simon, J. C., et al., J. Immunol. 146:485-491, 1991) were analysed by FACS. Human DCs were formed according to Sallusto, F. & Lanzavecchia; A., J. Exp. Med. 179:1109-1118, 1994, and contained 80-90% CDla⁺ cells.

### EXAMPLE 2

Immunostaining and flow cytometry

Cells were stained according to Weiss, J. M., et al. Eur. J. Immunol. 25:2858-2862, 1995, with one of the following antibodies: (a) human specific: N-terminal epitope=pan CD44, Leu 44 (clone L178, mouse IggGl, Becton Dickinson); exon v4, FW 11.10.3 (mouse IgGl, ECACC No. 93070776); exon v5, VFF8 (mouse IgGl, Bender, Vienna); exon v6, VFF18 (mouse IgGl, Bender, Vienna, WO 95/33771, DSM ACC2174); exon v7/v8, VFF17 (mouse IgG2b, Bender, Vienna); exon v9, FW 11.24.7.36 (mouse IgGl, ECACC No. 93070775). (b) mouse-specific: N-terminal epitope=pan CD44, IM7.8.1 (rat IgGl, ATCC Nr. TIB-235, Lesley, J. et al., Cell. Immunol. 112:40-54, 1988; Lesley, J. et al., Cell Immunol. 117:378-388, 1988; Trowbridge, I. S. et al., Immunogenetics 15:299-312, 1982; Budd, R. C. et al., J. Immunol. 138:3120-3129, 1987); a monoclonal antibody directed against exon v4; a monoclonal antibody directed against exon v6; isotype control, IB7 (rat IgGl; Dianova, Hamburg). Secondary antibodies: FITC-labelled sheep-anti-mouse (Fab)₂ and sheep-anti-mouse (Fab)₂ (Dianova, Hamburg). Treatment with the secondary antibody was followed by 10 minutes incubation in 2% normal mouse or rat serum followed by incubation with PE-(phycoerythrin)-labelled HLA-DR specific antibody (L234, mice IgGl, Becton Dickinson) or Iab (mice IgG2b, Pharmingen, Hamburg) or corresponding non-reactive PE-labelled control-antibodies (Dianova). Human DC was identified by FITC-conjugated mAb against CDla (Okt-6, mice IgGl, Ortho, Neckargemund). 7-Aminoactinomycin D (7-ADD) (2.5 mg/ml, Sigma) was added in order to exclude dead cells. Cell Quest Software (Becton Dickinson) was used to analyse 10⁴ HLA-DR⁺, CDla⁺ or Ia^{b} live cells.

### EXAMPLE 3

Full-thickness skin organ culture

4 mm thick skin-punch biopsies, containing dermis and epidermis were incubated in 25 mm tissue culture wells having a 0.02 mm Anopore Membrane (Nunc) in 6-well tissue culture dishes which had been filled with supplemented DMEM/HAMS-F12 (Gibco) up to the epidermo-dermal interface. Cultivations were ended at the specified times and samples were flash frozen in N₂. Tissue sections (5 mm) were dissected out (Cryocut 1800, Leica) and stained with an immunofluorescence double stain using the method of Negoescu, A. et al., J. Histochem. Cytochem. 42:433-437, 1994. First, frozen sections with Lag mAB were incubated for 30 minutes at ambient temperature and then at 4° C. overnight with FITC-conjugated goat-anti-mouse monovalent antibodies (Fab) (Dianova). Secondly, frozen sections were incubated with mAbs against CD44 epitopes for 30 minutes at ambient temperature and then with Cy3-conjugated goat-anti-mouse antibodies (Fab) (4° C., 4 hours). Cross-interference between the different staining steps was prevented by suitable controls. For quantitative analysis of total LC and CD44⁺/Lag⁺ double-positive LC, 5 greatly magnified areas were counted under the microscope and the number of LCs/mm² was determined.

### EXAMPLE 4

Split-thickness skin organ culture

2×2 cm Split-thickness skin containing epidermis plus papillary dermis was dissected using Dermatomes (Aesculap, Tuttlingen) according to Pope, M. et al., J. Invest. Dermatol. 104:11-17, 1995, and placed on supplemented RPMI in 6-well plates (Greiner, Nüurtingen). After 3 days, the cells which had migrated into the culture medium were collected. The keratinocytes were forced into a suspension by dermatome manipulation. Staining with FITC-conjugated mabs against CDla and FACS were carried out as described above. All the cells obtained from one well (5-6×10⁵) were analysed. The percentage of CDla⁺ cells was calculated using Cell Qest® Software (Becton Dickinson, Heidelberg). The percentage of emigrating LC from untreated samples was defined as 0% inhibition.

### EXAMPLE 5

Lymph node adhesion assay

LC or DC MACS (Miltenyi Biotec, Bergisch Gladbach) enriched with antibodies against HLA-DR or CDla, according to Simon, J. C. et al., Exp. Dermatol. 4:155-161, 1995, were tested for their binding to lymph node frozen sections (Pope, M. et al., J. Invest. Dermatol. 104:11-17, 1995). Fresh, frozen sections (10 mm) of human axillary lymph nodes placed on slides were blocked for 1 hour at 7° C. with RPMI containing 1% bovine serum albumin (BSA). LCs or DCs were suspended in HBSS (Gibco)/1%- BSA (2×10⁶ cells/ml) and the frozen sections were put on for 40 minutes at ambient temperature. The slides were rinsed with HBSS and fixed in acetone for 10 minutes at 4° C. In order to block the antibodies, LCs or DCs were pre-incubated with mAbs (in amounts of 20 mg/ml for 30 minutes at 4° C.) which were directed against the N-terminus of CD44 (SFF-2, MEM-85), with v exon specific antibodies (v5, VFF-8; v6, VFF-18; v9, FW 11.24.7.36), with ICAM-1 mAb 84H10 (Immunotech Corp., Boston, USA; Makgoba, M. W. et al., Nature 331:86-88, 1988) or with control IgGl. The slides were then stained with anti-CDla mAB according to Simon, J. V. C. et al., Europ. J. Cancer 32A:1394-1400, 1996. Coding and evaluation were carried out by two independent researchers. The background-binding of cultivated LC/DC was roughly 1.5 cells/mm², whilst the positive-binding was 30 cells/mm². The data were recorded as % binding compared with samples having no antibody. The standard deviation was calculated using three slides, each with 9 random areas per slide, using an optical lattice (magnification 10x).

### EXAMPLE 6

Contact hypersensitivity

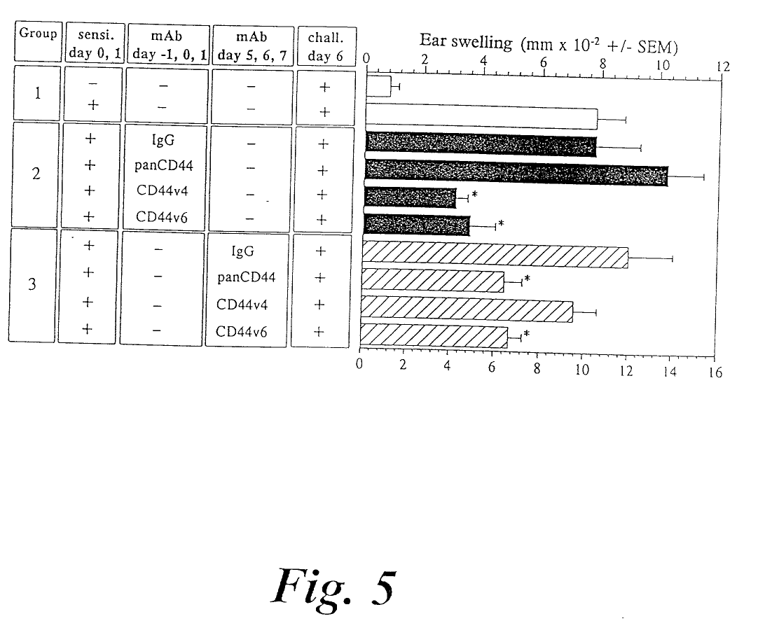

6 Week old female C57/BL63 mice (Harlan-Olac, Great Britain) had 20 μl of 0.5% 2,4-dinitrofluorobenzene (DNFB; Sigma, Deisenhofen) in acetone applied to their abdominal skin on day 0 and 1 according to Simon, J. C. et al., Photodermatol. Photoimmunol. Photomed. 10:206-211, 1994. On day 6, 10 μl of 0.2% DNFB was applied to both sides of the right ear of each mouse. The thickness of the ear was measured with Engineer's Micrometer (Mitutoyo Corp., Takatsu-Ku, Kawasaki-Shi, 213 Kanagawa-Ken, Japan) before and 24 hours after the challenge (Simon, J. C. et al., Photodermatol. Photoimmunol. Photomed. 10:206-211, 1994). The group 1 mice were only challenged or sensitised and challenged in the absence of mAbs. The mAbs were injected (i.p.) as follows: group 2 mice were given 300 mg on day 1, 100 mg on days 0 and 1. Group 3 mice were given 300 mg on day 5 and 100 mg on days 6 and 7. Each bar (FIG. 5) represents the average values of the ear swellings pooled from 8 animals.

1. Use of anti-sCD44 and/or anti-vCD44 antibodies for producing a preparation for the prophylactic and therapeutic treatment of malignant diseases which are associated with the degeneration, activation or massive multiplication of Langerhans cells (LC) and/or dendritic cells (DC).

2. Use of anti-sCD44 and/or anti-vCD44 antibodies for producing a preparation for the prophylactic and therapeutic treatment according to claim 1, characterised in that the malignant diseases are Langerhans-cell-histiocytoses, histiocytosis X, Abt-Letterer-Siewe syndrome or eosinophilic granuloma.

3. Use of anti-sCD44 antibodies for producing a preparation for the prophylactic and therapeutic treatment according to claims 1 and 2, characterised in that the antibodies recognise N-terminal epitopes of sCD44, or parts thereof.

4. Use of anti-vCD44 antibodies for producing a preparation for the prophylactic and therapeutic treatment according to claims 1 to 3, characterised in that the antibodies recognise epitopes coded by variant exons of vCD44, or parts thereof.

5. Use of anti-vCD44 antibodies for producing a preparation for the prophylactic and therapeutic treatment according to claim 4, characterised in that the antibodies recognise epitopes coded by the variant exons v4, v5, v6 and/or v9, or parts thereof.

6. Use of anti-vCD44 antibodies for producing a preparation for the prophylactic and therapeutic treatment according to claim 5, characterised in that the antibodies are capable of reacting with the following amino acid sequences or parts thereof:
ISTTPRAFDHTKQNQDWTQWNPSHSNPEVLLLQTTTRMT
DVDRNGTTAYEGNWNPEAHPPLIHHEHHEEEETPHSTST
QATPSSTTEETATQKEQWFGNRWHEGYRQTPREDSHSTTGTA
QQSNSQSFSTSHEGLEEDKDHPTTSTLTSS.

7. Use of anti-vCD44 antibodies for producing a preparation for the prophylactic and therapeutic treatment according to claim 6, characterised in that the antibody consists of the antibody VFF-18 directed against the epitope coded by v6, or parts thereof, and those antibodies which are capable of reacting with the epitope recognised by VFF-18 or parts thereof.

8. Use of anti-sCD44 antibodies for producing a preparation for the prophylactic and therapeutic treatment of diseases and conditions of a mammalian organism based on an immunoregulatory disorder or an undesirable or excessive immune reaction.

9. Use of anti-sCD44 antibodies for producing a preparation according to claim 10, characterised in that the immunoregulatory disorder or the undesirable or excessive immune reaction are allergic diseases, allergies of the delayed type, rejection of skin or organ transplants, autoimmune diseases, diseases of the rheumatic type, multiple sclerosis, psoriasis or atopical dermatitis.

10. Use of anti-sCD44 antibodies for producing a preparation for the prophylactic and therapeutic treatment according to claims 8 and 9, characterised in that the antibodies recognise N-terminal epitopes of sCD44, or parts thereof.

11. Use of anti-sCD44 and/or anti-vCD44 antibodies for producing a preparation for the prophylactic and therapeutic treatment according to claims 1 to 10, characterised in that the antibodies are monoclonal antibodies or fragments or derivatives thereof.

12. Use of the antibody VFF-18 directed against the epitope coded by v6, or parts thereof, and those antibodies which are capable of reacting with the epitope recognised by VFF-18 or parts thereof, for the prophylactic and therapeutic treatment of diseases and conditions of mammalian organisms according to one of claims 8 and 9.

13. Process for producing dendritic cells, characterised in that isolated monocytes are cultivated for a few days in a culture medium consisting of RPMI 1640, foetal calf serum, penicillin/streptomycin, a buffer consisting of an N-substituted aminosulphonic acid, non-essential amino acids, L-glutamine, GM-CSF and an interleukin, and then isolated.

14. Process for producing dendritic cells according to claim 13, characterised in that the isolated monocytes are cultivated for 8 days in a culture medium consisting of RPMI 1640, 10% FCS, 45 μ penicillin/streptomycin, 25 mM Hepes, 1 mM non-essential amino acids, 2 mM L-glutamine, 50 ng/ml human GM-CSF and 1000 U/ml IL-4 and then isolated.


SEQUENCE LISTING (4 sequences)

SEQ ID NO: 1 - protein, 39 aa, Homo sapiens
ISTTPRAFDHTKQNQDWTQWNPSHSNPEVLLLQTTTRMT

SEQ ID NO: 2 - protein, 39 aa, Homo sapiens
DVDRNGTTAYEGNWNPEAHPPLIHHEHHEEEETPHSTST

SEQ ID NO: 3 - protein, 42 aa, Homo sapiens
QATPSSTTEETATQKEQWFGNRWHEGYRQTPREDSHSTTGTA

SEQ ID NO: 4 - protein, 30 aa, Homo sapiens
QQSNSQSFSTSHEGLEEDKDHPTTSTLTSS